(19) United States
(12) Patent Application Publication
(10) Pub. No.: US 2021/0391084 A1
(43) Pub. Date: Dec. 16, 2021

(54) CLINICAL SMART WATCH FOR ADDRESSING ADVERSE CARDIAC EVENTS

(71) Applicant: Rhonda Fay Adams, Braintree, MA (US)

(72) Inventor: Rhonda Fay Adams, Braintree, MA (US)

(21) Appl. No.: 17/285,518

(22) PCT Filed: Oct. 15, 2019
(86) PCT No.: PCT/US2019/056220

§ 371 Date: Apr. 15, 2021

Related U.S. Application Data
(60) Provisional application No. 62/745,525, filed on Oct. 15, 2018.

(51) Int. Cl. G16H 50/30; G16H 40/63; G06F 1/16

(52) U.S. Cl. CPC G16H 50/30; G06F 1/163; G16H 40/63

(57) ABSTRACT

A clinical smart watch, comprises a housing; a band coupled to the housing, at least one of the housing or the band including one or more sensors that measure health-related events of a wearer of the smart watch; a cardiac event risk assessment processor in the housing that receives and processes sensor signals corresponding to the health-related events; and a user interface that includes an the sensor signals, a second risk score generated from a second combination of the user input data and the sensor signals, and a third risk score generated from a third combination of the user input data and the sensor signals. input for providing user input data to the cardiac event risk assessment processor. The cardiac event risk assessment processor executes a prediction model including a first risk score generated from a first combination of the user input data and

### RELATED APPLICATIONS

This application claims priority to United States provisional patent application Ser. No. 62/745,525, filed Oct. 15, 2018 and entitled “SMART WATCH,” the contents of which are incorporated herein by reference in its entirely.

### TECHNICAL FIELD

The present inventive concepts generally relate to systems, methods, and wearable devices for processing health care information, and more particularly, to a smart watch that proactively detects a cardiac health event and performs various operations directed to addressing the cardiac health event.

### BACKGROUND

Modern smart watch technology can receive and process data from various external sources for display on a touchscreen interface. Accordingly, smart watches can perform the functions of many different electronic devices such as a multi-media player, navigation device, calculator, personal organizer, and so on.

One emerging smart watch application relates to personal health, where a smart watch can display in real-time clinical information such as heart rate or electrocardiogram (ECG) information. However, these smart watches only collect and report information, and provide no features that proactively determines whether a medical emergency is present and properly communicating the medical emergency with respect to a possible cardiac event such as a stroke, myocardial infarction, angina coronary artery bypass grafting, percutaneous coronary intervention, and so on.

### SUMMARY

In one aspect of the present inventive concepts, provided is a clinical smart watch, comprising: a housing; a band coupled to the housing, at least one of the housing or the band including one or more sensors that measure health-related events of a wearer of the smart watch; a cardiac event risk assessment processor in the housing that receives and processes sensor signals corresponding to the health-related events; a user interface that includes an input for providing user input data to the cardiac event risk assessment processor, wherein the cardiac event risk assessment processor executes a prediction model including a first risk score generated from a first combination of the user input data and the sensor signals, a second risk score generated from a second combination of the user input data and the sensor signals, and a third risk score generated from a third combination of the user input data and the sensor signals; a peripheral device for initiating an electronic communication with a remote electronic device when at least one of the first, second, or third risk scores exceeds a threshold value indicating a probability of an adverse cardiac event; a distress button that activates the peripheral device to initiate the electronic communication with the remote electronic device; a medication compartment that stores a source of medication for treating the adverse cardiac event; and a display for displaying each of the first, second, and third risk scores, and for generating a visual alert when the at least one of the first, second, or third risk scores exceeds the threshold value.

In another aspect, a clinical smart watch, comprises one or more computing devices; and non-transitory memory medium that stores instructions that when executed by the one or more computing devices executes the clinical smart watch to perform at least one operation comprising the steps of: collecting tri-measurement score data from a combination of sensors, health status repository, and user inputs; generating a tri-measurement score, comprising: generating a HEART (History, ECG (EKG), Age, Risk actors, and Troponin) risk score; generating a GRACE (Global Registry of Acute Coronary Events) score; and generating a TIMI (Thrombolysis in Myocardial Infarction) score; comparing the tri-measurement score to a threshold value; and generating a cardiac event assessment notification in response to a result of the comparison of the tri-measurement score and the threshold value.

In another aspect, a clinical smart watch, comprises one or more computing devices; and a non-transitory memory medium that stores instructions that when executed by the one or more computing devices executes the clinical smart watch to perform at least one operation comprising the steps of determining a plurality of cardiac indicators, including: generating a first user interface display in response to a determination that all of the cardiac indicators exceed a risk threshold; generating a second user interface display in response to a determination that a majority of the cardiac indicators exceed a risk threshold generating a third user interface display in response to a determination that a minority of the cardiac indicators exceed a risk threshold; and generating a fourth user interface display in response to a determination that none of the cardiac indicators exceeds a risk threshold.

### BRIEF DESCRIPTION OF THE DRAWINGS

The foregoing and other objects, features and advantages of embodiments of the present inventive concepts will be apparent from the more particular description of preferred embodiments, as illustrated in the accompanying drawings in which like reference characters refer to the same elements throughout the different views. The drawings are not necessarily to scale, emphasis instead being placed upon illustrating the principles of the preferred embodiments.

FIG. 1 is a perspective view of a smart watch, in accordance with some embodiments;

FIG. 2 is a block diagram of a printed circuit board (PCB) of the smart watch of FIG. 1, in accordance with some embodiments;

FIG. 3 is a schematic diagram of a smart watch operating in a network environment, in accordance with some embodiments;

FIG. 4 is another view of a user interface display of a smart watch, in accordance with some embodiments;

FIG. 5 is a flow diagram of a method for processing cardiac for proactive purposes, in accordance with some embodiments;

FIG. 5A is a block diagram of the cardiac event risk assessment processor of the smart watch of FIGS. 1 and 2, in accordance with some embodiments.

FIG. 6 is a flow diagram of an operation performed by a smart watch in response to the selection of a call button displayed at the smart watch, in accordance with some embodiments;

FIG. 7 is a flow diagram of an operation performed by a smart watch in response to the selection of an emergency contact button displayed at the smart watch, in accordance with some embodiments;

FIG. 8 is a flow diagram of an operation performed by a smart watch in response to the selection of a vital signs button displayed at the smart watch, in accordance with some embodiments;

FIG. 9 is a flow diagram of an operation performed by a smart watch in response to the selection of a health history button displayed at the smart watch, in accordance with some embodiments;

FIG. 10 is a flow diagram of an operation performed by a smart watch in response to the selection of an EEG/PPG information button displayed at the smart watch, in accordance with some embodiments;

FIG. 11 is a flow diagram of an operation performed by a smart watch in response to the selection of a blood clot information button displayed at the smart watch, in accordance with some embodiments;

FIG. 12 is a flow diagram of an operation performed by a smart watch in response to the selection of a heart assessment button displayed at the smart watch, in accordance with some embodiments;

FIG. 13 is a flow diagram of a method for assessing a cardiac event, in accordance with some embodiments;

FIG. 14 is a flow diagram of an operation performed by a smart watch in response to the selection of a medication dispensing button displayed at the smart watch, in accordance with some embodiments;

FIGS. 15-19 are examples of various data entry screens, in accordance with some embodiments;

FIGS. 20-24 are examples of database records including various medical data processed by a smart watch, in accordance with some embodiments;

FIG. 25 is an example of a HEART (History, ECG (EKG), Age, Risk factors, and Troponin) scoring matrix, in accordance with some embodiments;

FIGS. 26A and 26B are examples of a GRACE (Global Registry of Acute Coronary Events) scoring matrix, in accordance with some embodiments; and

FIG. 27 is an example of a TIMI (Thrombolysis In Myocardial Infarction) risk scoring matrix, in accordance with some embodiments.

### DETAILED DESCRIPTION OF EMBODIMENTS

The terminology used herein is for the purpose of describing particular embodiments and is not intended to be limiting of the inventive concepts. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements, and/or components, but do not preclude the presence or addition of one or more other features, integers, steps, operations, elements, components, and/or groups thereof.

It will be understood that, although the terms first, second, third etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on” or “connected” or “coupled” to another element, it can be directly on or above, or connected or coupled to, the other element or intervening elements can be present. In contrast, when an element is referred to as being “directly on” or “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.). When an element is referred to herein as being “over” another element, it can be over or under the other element, and either directly coupled to the other element, or intervening elements may be present, or the elements may be spaced apart by a void or gap.

Conventional smart watches, including those provided by Apple, Fitbit, and so on, can include various software applications that collect, store, and display medical information such as a heart rate or ECG signal, which can inform a user in real-time of possible irregularities. However, such devices do not, and cannot, proactively determine the probability of a heart attack, or other medical conditions such as arrhythmias, atrial fibrillations, hypertension and thrombosis, because the ECG is one or a multitude of different tests and cannot per se predict a heart attack. A high ECG or heart rate may be one factor of a possible heart attack, but a high ECG or heart rate reading could also be taken while the smart watch wearer is jogging or performing a stress-related activity, which is unrelated to the risk of a heart attack.

In brief overview, the inventive concepts include a clinical smart watch that prevents and/or delays adverse cardiac events for patients who have the potential of a cardiac event, such as a stroke, heart attack, etc. The smart watch provides effective and efficient health care information to cardiac patients via a 24 hour monitoring system that is user friendly and allows collaborative efforts to be established between computer-related devices of a patient, emergency contact representatives, and/or medical staff. The smart watch relies on a set of medical/clinical scoring protocols constructed and arranged as a prediction model to determine possible adverse cardiac events based on a detection of a plurality of specific indicators, which are processed to determine the potential for an adverse cardiac event, and performs actions directed at preventing the cardiac event from occurring if the potential is greater than a predetermined threshold. A cardiac event may be one of the abovementioned events such as a stroke heart, myocardial infarction, angina coronary artery bypass grafting, or percutaneous coronary intervention, but not limited thereto. In some embodiments, the clinical smart watch when in operation continuously collects, monitors, and displays personal health care information, and further processes this information to supply the wearer with customized health information that pre-warns or cautions adverse cardiac health events. To achieve this, the smart watch when worn provides a unique tri-measurement scoring feature that triggers mechanical, electrical, and/or software features in response to historical and real-time inputs in combination to provide a risk assessment of a possible cardiac event, and to execute an action directed at preventing an occurrence of a dangerous or otherwise life-threatening cardiac event. This feature is different from conventional smart watches, which process medical information so that a wearer can react to a potential negative health risk, but are incapable of proactively reporting potentially harmful cardiac health events. More specifically, embodiments of the inventive clinical smart watch when worn continuously is configured to function as a real-time indicator of a health status based on multiple real-time inputs and stored historical information. In doing so, the inventive smart watch processes collected medical data in a manner that can proactively report potentially harmful cardiac health events to emergency and medical staff and allow for an immediate diagnosis and/or determination of immediate options for treatment and intervention development. This provides heightened healthcare awareness and increased personal knowledge of cardiac conditions, e.g., strokes, heart attacks, and so on, in some cases including inputs from artificial intelligence technology and/or analytics technology that connect to patient/and or healthcare agents to engage in a proactive decision making process

The foregoing features are advantageous over conventional smart watches, which are reactionary in that they provide results in response to an event, and do not proactively report cardiac-related abnormalities by continuously monitoring the patient based on proactive inputs, for example, current data from recent clinical data, and also determining multiple risk sources to determine whether an alert or other response is warranted, such as outputting automatically an alert to medical personnel or other emergency resource.

Accordingly, a smart watch in accordance with the inventive concepts provides a consumer with a reliable, dependable tool that provides an expeditious response to potential adverse cardiac events. In some embodiments, the inventive technology is combined with medication therapy to track its effectiveness to stabilized and/or abnormal readings, permitting the smart watch to assist providers with home medication dispensing, patient history, current/abnormal vital signs, patient contacts, blood clot detection, and provide medical personnel an advanced notice regarding possible treatments/interventions that should be prescribed immediately.

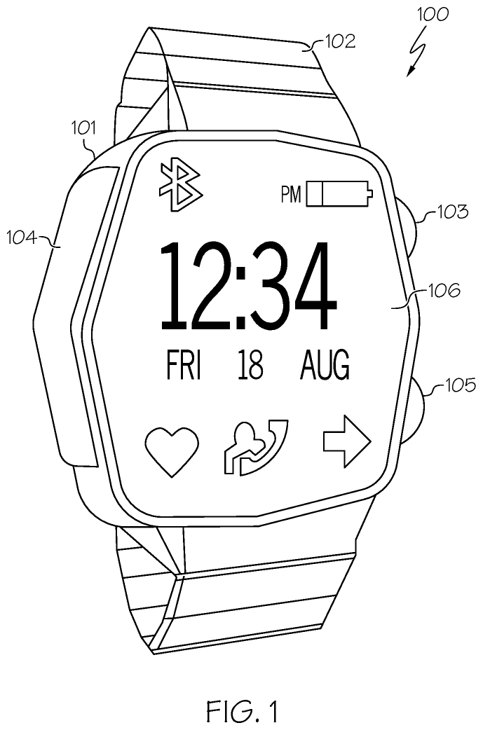

FIG. 1 is a perspective view of a smart watch 100, in accordance with some embodiments. The smart watch 100 may include but not be limited to a housing 101, a band 102, a reset or distress button 103, a medication storage compartment 104, and a display 106 that presents a user interface executed by a special purpose processor of the smart watch. These elements may interoperate to provide a water-resistant watch.

The band 102 is coupled to the housing 101 and can include a buckle or other elements for securely wrapping around the wearer's wrist. Preferred embodiments include use by a human wearer, but the smart watch 100 may in other embodiments be worn by an animal such as a pet. In some embodiments, the band 102 includes one or more sensors, modules, accelerators, gyroscopes, or the like that directly touch or are in close proximity to the wearer's body to gather data regarding the wearer such as heart beat/heart rate/pulse, electrocardiogram (ECG), photoplethysmography (PPG), and so on. Other embodiments include electronic smart devices that do not have a watchband but instead have a clasp or other coupling element for attaching to a part of the human body. In other embodiments, such sensors, modules, or the like, are positioned on a surface of the housing 101 of the watch 100 in addition to or instead of the band 102. In some embodiments, sensors such as heart rate, ECG, and so on, are wireless sensors and in wireless communication with the various processors in the housing 101. Example sensors may include but not be limited to sensors that measure heart rate, arterial pressure, glucose, temperature, weight, oxygen, sweat, skin conduction, arterial pressure, physiological conditions, and so on. In some embodiments, the band 102 is formed of various materials that are resistant to allergies, i.e. allow allergy-sensitive users to wear the smart watch 100 in a practical and safe manner.

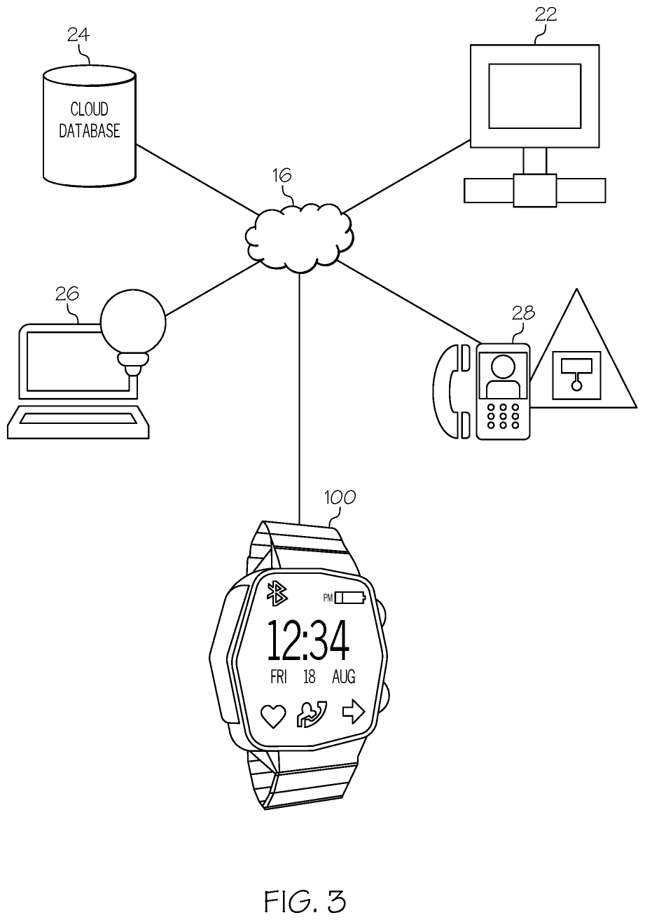

The reset button 103 is constructed and arranged to refresh a current display score and warnings, for example, described below. In some embodiments, the reset button 103 when activated also initiates a distress communication (voice, SMS text, and so on) to a predetermined emergency contact, 911 operator, and/or other communication device 28 (see FIG. 3).

The medication storage compartment 104 may be part of the housing 101 or removably extend from or otherwise be coupled to the housing for storing medication such as pills prescribed by a physician. The medication storage compartment 104 can store a source of medication such as pills, and be activated, i.e., opened or closed, by applying a force by a user's finger or object applied directly to the storage compartment 104 or to a release button 105 to cause a spring to open the storage compartment 104 so that pills or the like can be placed in it, then closed by the user's finger or object. In some embodiments, the storage compartment 104 is opened automatically in response to a call made from the smart watch 100 to an emergency contact, hospital, first responder, and so on to alert of medication use. In some embodiments, the opening and/or closing of the storage compartment 104 can trigger messages to be output, for example, automatically sending via text/email, or the like one or more risk assessment data such as abnormal measurements to an emergency contact or other destination electronic device identified by a contact stored in memory of the smart watch 100.

The medication compartment 104 may have a locking mechanism and his constructed and arranged to expose its contents, e.g., pills, in response to a determination of a tri-measurement score (described herein), which in turn provides for a special-purpose processor to automatically control the locking mechanism to open the medication compartment, activate gears to withdraw the compartment 104 from the housing 101 or otherwise render its contents, e.g., pills, available to the user. In some embodiments, a button 105 activates the medication compartment so that the user can control the opening and closing of the medication compartment after reviewing a tri-measurement score for other reasons.

The electronic display 106 may be part of the housing 101 or extend from or otherwise be coupled to the housing for displaying digital information and or visual information of data and/or messages, images, and/or other visual, audio, and/or tactile information to the user. The display 106 may include a touchscreen or the like. The display may be scratch-resistant, water-proof, or related feature.

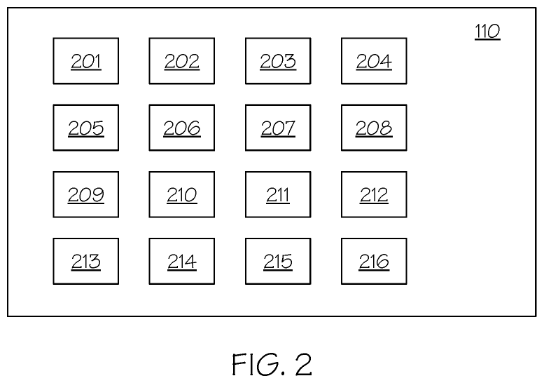

FIG. 2 is a block diagram of a printed circuit board (PCB) 110 of the smart watch of FIG. 1, in accordance with some embodiments.

In some embodiments, the PCB 110 includes some or all of, but is not limited to, a battery 201, a Wi-Fi signal processor 202, a global positioning system (GPS) signal processor 203, a cell phone processor 204, an audio device 205, one or more sensor processors 206, a computer memory 207, a wireless communication device 208, a cardiac event risk assessment processor 209, a universal serial bus (USB) connector 210, an on/off power button 211, a speaker 212, a microphone 213, a touchscreen display processor 214, a phone pairing device 215, and a light source 216.

The battery 201 powers the other PCB components as well as the electrical and electro-mechanical components of the smart watch 100.

The Wi-Fi signal processor 202, GPS signal processor 203, and cell phone processor 204 (collectively referred to as a communication processor) each processes signals exchanged with external computers via an antenna (not shown). The antenna for the Wi-Fi signal processor 202, GPS signal processor 203, and cell phone processor 204 may be the same or different. The features of the Wi-Fi signal processor 202, GPS signal processor 203, and cell phone processor 204 may be the same or different. For example, the GPS signal processor 203 may receive and process user location data, and output a result to the display 106 while the cell phone processor 204 outputs and receives incoming and outgoing calls and text messages. The wireless communication device 208 can include one or more computer processors and an antenna or the like to connect smart watch and external communication devices, either directly, for example, a Bluetooth™ connection or the like, or via the network 16, electronic pairing to a smartphone such as an Android™ or iPhone™ device. Alternatively, or in addition, the phone pairing device 215 includes electronic components and the antenna, or a peripheral interface for a conductive wire to extend to another phone or to a network 16 which in turn can form communications between the smart watch 100 and another phone. In some embodiments, the wireless communication device 208 communicates with wireless sensors or wired sensors in the housing or band of the smart watch 100 that provide relevant input data for producing a cardiac event risk assessment, described herein.

The audio device 205 can include a speaker or the like for outputting verbal information, tones, or any other sounds for communicating with the user. Other devices such as tactile or haptic feedback or vibration mechanisms may be part of the smart watch 100 for communicating information to the user in addition to or alternatively to the visual display 106 or audio device 205.

The sensor processors 206 process signal data detected by the various sensors or modules of the smart watch 100 such as heart rate, ECG, PPG, body temperature, body fluids, and so on. For example, a sensor processor 206 can process collected information regarding a heart rhythm using the optical heart sensor of the smart watch 100. This data can be stored at the computer memory 207 or the like, for example, described below as computer readable storage medium, or other data storage device as executable software codes that are executed by one or more processors or other computer hardware described herein. In some embodiments, a sensor processor 206 can continuous measure a user's physiological status. For example, a sensor processor 206 can process collected information regarding a heart rhythm using the optical heart sensor of the smart watch 100. This data can be stored at the computer memory 207 or the like, for example, described below as computer readable storage medium, or other data storage device as executable software codes that are executed by one or more processors or other computer hardware described herein. In some embodiments, a sensor processor 206 can continuous measure a user's physiological status. In some embodiments, the memory 207 can store log reports, including data for display, such as alerts sent, device error messages, emergency contact messages sent, and so on. In some embodiments, this data is exchanged with a cloud database 24 for storing this data and/or other health status information.

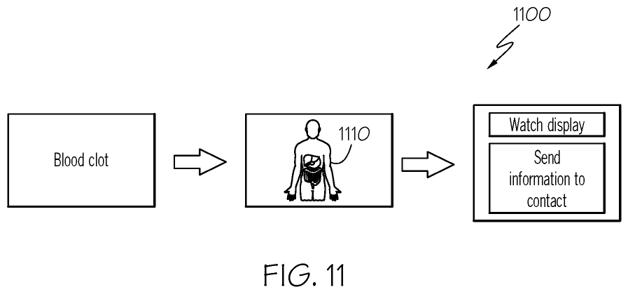

The light source 216 is used to emit light through blood vessels to obtain PPG measurements, for example, described with respect to an example application in FIG. 11.

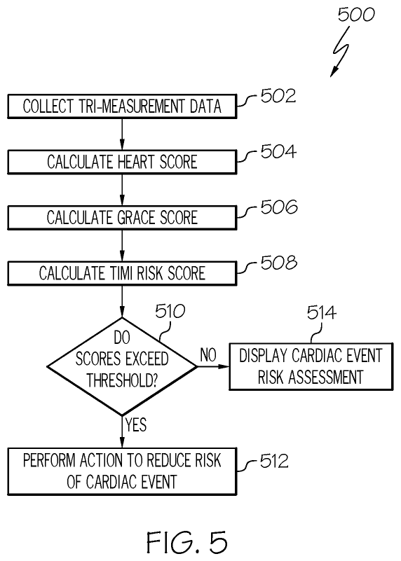

The cardiac event risk assessment processor 209 generates a “score” that provides a risk assessment of a possible cardiac event, which can be used to determine an action directed at prevent an occurrence of a dangerous or otherwise life-threatening cardiac event. Details of the cardiac event risk assessment processor 209 are described at FIGS. 5 and 5A.

FIG. 3 is a schematic diagram of a smart watch 100 operating in a network environment 16, in accordance with some embodiments. The smart watch 100 is constructed and arranged to communicate with one or more of a third party computer 22, a cloud database 24, an internet computer location 26, and/or an emergency contact 28 via a network 16. The network 16 can be any communication network(s) such as a wired and/or wireless data communication network.

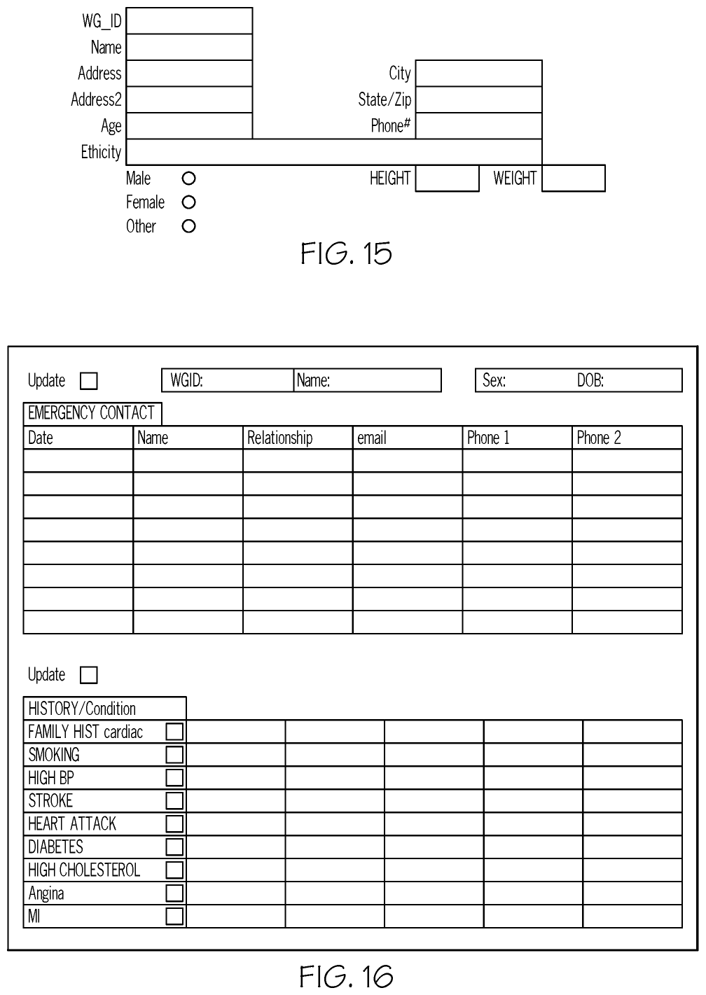

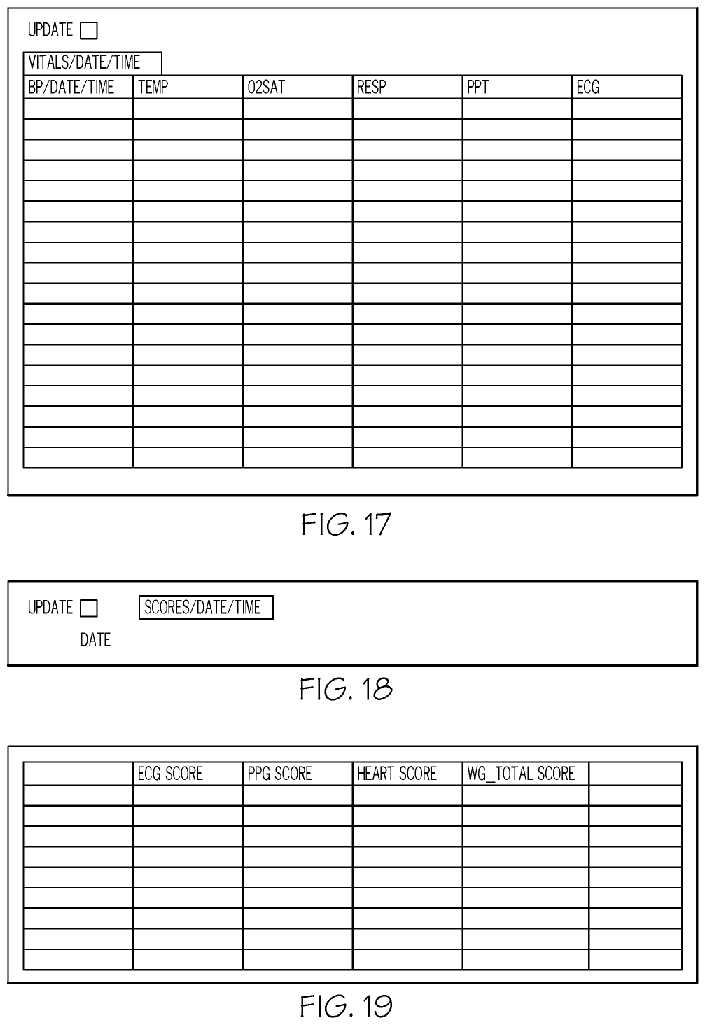

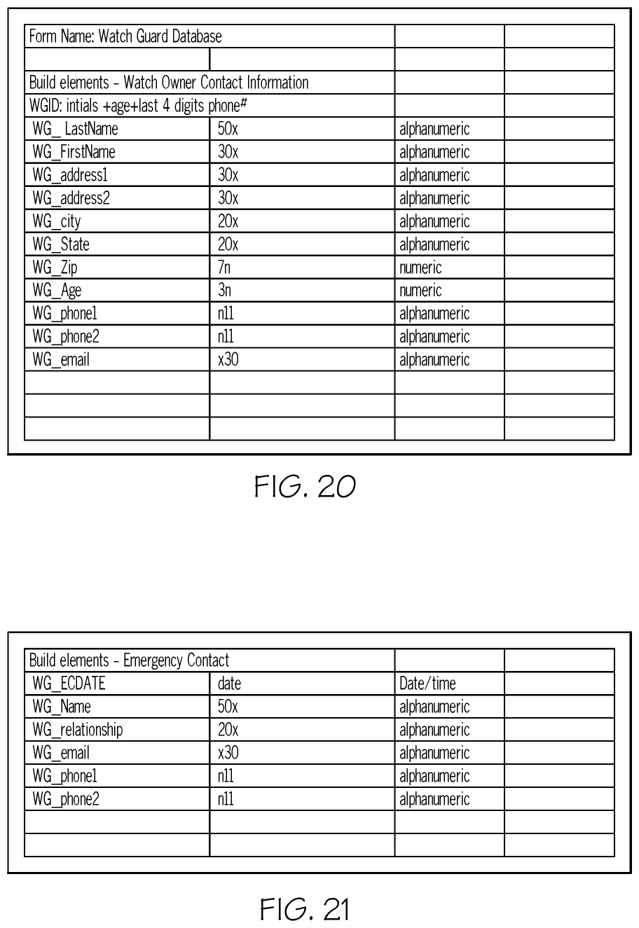

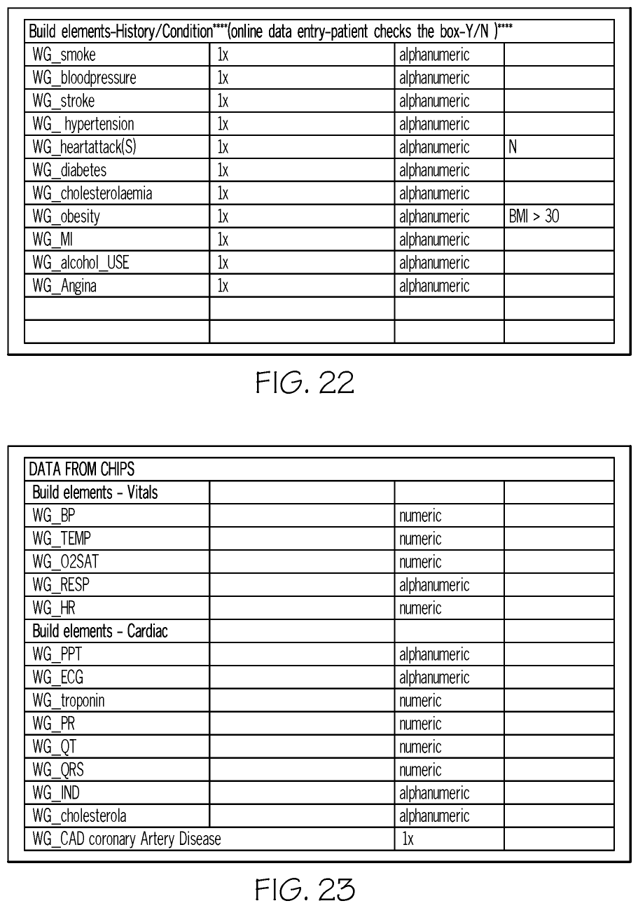

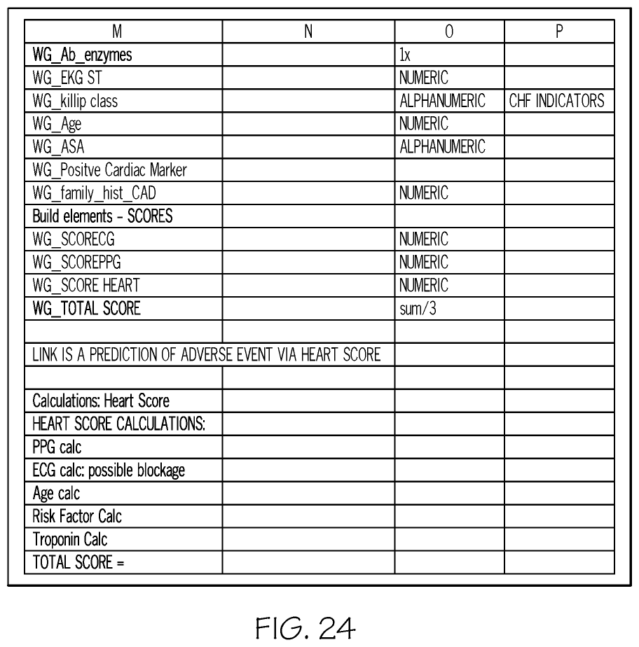

The cloud database 24 is configured to serve as a repository for storing patient data, such as health history, health status, clinical, and/or other information for producing a prediction model with respect to cardiac event assessments. Data of the prediction model can be provided to the smart watch 100 for executing operations according to the model. Medical alert data can be entered by a user or machine, for example, at a computer location 26 in a secured manner for storage in the database 24. Examples of user inputs are illustrated in FIGS. 15-19, which can be stored in memory, for example, at the database 24 for subsequent retrieval by the smart watch 100 and/or stored in computer memory 207 of the smart watch 100. Information can be retrieved from the database 24 and viewed at the display 106 of the smart watch 100 and/or a remote computer location 26, such as a secured “You Take Control” (YTC) website. To maintain security, the cloud database 24 may store and process data protection software, blockchain security, biometrics, and so on. The internet computer location 26 can provide a data entry screen for a user to enter health care data which can be stored at the database 24 and/or received by the smart watch 100. Accordingly, the smart watch 100 can exchange response data or the like with the cloud medical database 24.

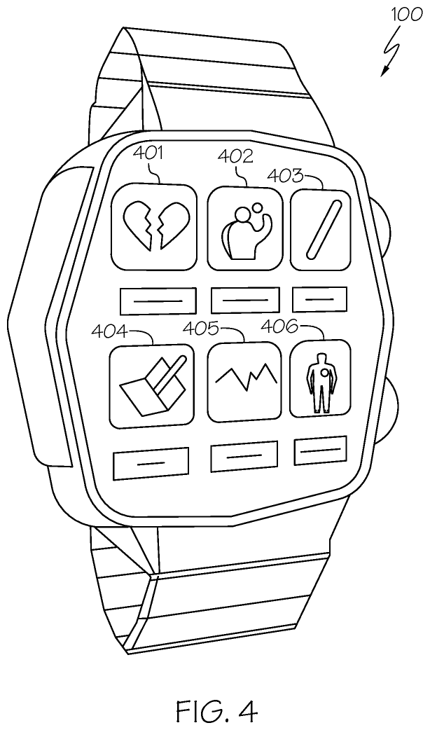

FIG. 4 is another view of a user interface display of the clinical smart watch 100 of FIGS. 1-3, in accordance with some embodiments. In particular, FIG. 4 illustrates a set of secondary functions performed by the clinical smart watch 100, distinguished from FIG. 1 which illustrates a displayed set of primary functions. Examples of the primary display may include but not be limited to a Wi-Fi IFI signal display, battery indicator (displays battery level/power), time/date, distress/reset button (via icon in addition to or as an alternative to physical reset button 103 shown in FIG. 1) when pressed can cause the smart watch 100 to call 911 and call emergency contact, HEART score output, abnormal health information output, and reset confirm button, which when pressed 3 times will ask for confirmation to reset to main screen display). Patient information can be view just by pressing the options listed and can also be used for emergency actions (intervention) as patient past conditions, allergies, and home medication can be viewed for effective treatments. Current vital sign, ECG, PPG, or related data can also be viewed when pressing the appropriate option by an output generated by the touchscreen display processor 214.

A displayed heart alert icon 401, or button or other activatable display on a secondary user interface display screen can be selected by a user touching the icon 401 on the display 106. Other modes of activating or selecting various icons on the display may be achieved by voice activation, or speech-to-data conversion technology of the smart watch 100. Regardless of selection mode, the heart alert icon 401 is activated and subsequently a current HEART score is displayed.

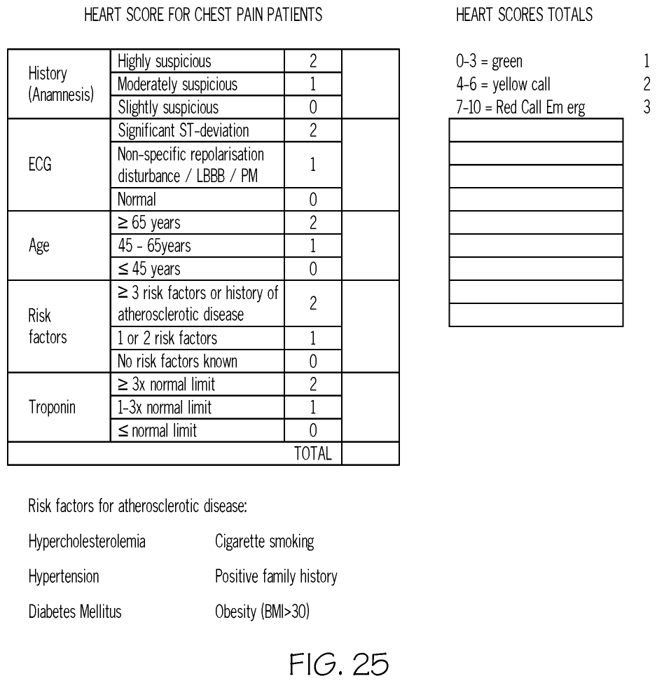

The current HEART score is a predictor of a major cardiac event such as a heart attack, and outputs a range of points well-known to those in the medical community, in particular, emergency room settings. In some embodiments, a HEART score is generated and output by the smart watch 100. For example, a 0 value for each HEART factor (age, ECG status, etc.) indicates a low score, a +1 value indicates a moderate risk, and a +2 value indicates a high risk. Although a value range of 0-2 is illustrated by way of example, other value ranges can equally apply. As described herein, embodiments of the present inventive concepts include a cardiac event risk assessment processor 209 that generate a HEART score including an aggregate of event values (each ranging from 0-2). An example is illustrated at FIG. 25, where a combined HEART score of 0-3 (combination of HEART events) results in the smart watch 100 generating a display warning or other visual, tactile output, a score of 4-6 results in an automatic call to an emergency contact, and a score of 7-10 indicates a high risk of a cardiac event and that a 911 call or other high alert is generated. Other actions in addition to or alternatively to an automatic call may be performed by the smart watch 100, such as text messages in lieu of a call, visual, audio, and/or tactile feedback generated at the smart watch 100, and so on.

A call icon 402, or button or other activatable display on the secondary screen when selected transitions the secondary user interface display screen to a user interface display screen that permits the user to place phone calls, text messages using short message service (SMS), or other communications, which can be output via the Wi-Fi, cell phone processor, and/or other input/output device of the smart watch 100.

A vital signs icon 403, or button or other activatable display on the secondary screen when selected displays vital sign information, and can further identify vital signs that have an abnormal or otherwise undesirable value, for example, a blood pressure reading displayed as “BP 180/90” which is outside the range of a “normal” blood pressure range.

A “problems” icon 404, or button or other activatable display on the secondary screen when selected displays information of possible concern to a user such as a current medical issue, home medication information, allergy warnings, and so on.

An ECG/PPG icon 405, or button or other activatable display on the secondary screen when selected displays ECG and/or PPG readings.

A clot icon 406, or button or other activatable display on the secondary screen when selected displays an approximate blood clot location.

FIG. 5 is a flow diagram of a method 500 for processing cardiac for proactive purposes, in accordance with some embodiments. In describing the method 500, reference is made to FIGS. 1-4. FIG. 5A is a block diagram of the cardiac event risk assessment processor 209 of FIG. 2, in accordance with some embodiments. Accordingly, some or all of the method 500 can be performed by elements of FIGS. 1-4 and 5A.

At block 502, three different sets of data, referred to as tri-measurement data, are collected by a combination of sensors and/or data inputs via the network interface. Tri-measurement data may be used to calculate one or more of a HEART score, a GRACE score, and a TIMI score, which can collectively provide a combined or total score used to predict a risk of a cardiac event such as a heart attack.

At block 504, a first special purpose computer processor 551 of the cardiac event risk assessment processor 209, referred to as a HEART score processor, can process a set of input data for the HEART score which may include a combination of real-time sensor data, historical data, medical opinions, age, risk factors, and/or lab results. At least one of the foregoing is received as an input by a sensor of the smartwatch that is processed by a sensor processor 206, such as an ECG sensor processor. The HEART score can therefore provide a prediction of combined evidence of myocardial infarction, percutaneous coronary intervention, and/or coronary artery bypass grafting, but not limited thereto.

At block 506, a second special purpose computer processor 552 of the cardiac event risk assessment processor 209, referred to as a GRACE score processor, generates a GRACE score from a second set of input data. A GRACE score is a tool used in the medical community that estimates a six month mortality prognosis for patients with acute coronary syndrome. A GRACE Risk Score having a value of 8, for example, may indicate the presence of blocked blood flow in the wearer of the smart watch 100, or more specifically to risk stratify patients with diagnosed ACS (blocked blood flow) to estimate the wearer's in-hospital and 6-month to 3-year mortality. In some embodiments, a GRACE Risk Score considers eight factors to independently predict risk of heart attack and/or death; such factors including age, heart rate, systolic blood pressure, renal function, congestive heart failure, ST-segment deviation, cardiac arrest, elevated biomarkers, and the like.

A GRACE score can range from 0-363 points. Points can be provided based on age, heart rate, systolic blood pressure, initial serum creatinine, and/or Killip class. For example, a 90 year old patient is provided 100 points, a heart rate of 200 beats per minute is provided 46 points, a systolic blood pressure of 160-199 mmHG is provided 10 points, a creatinine reading of 4.00 is provided 28 points, and a Killip class IV is provided 59 points. At least one of the foregoing is received as an input by a sensor of the smartwatch that is processed by a sensor processor 206, such as a blood pressure and/or heart rate sensor processor.

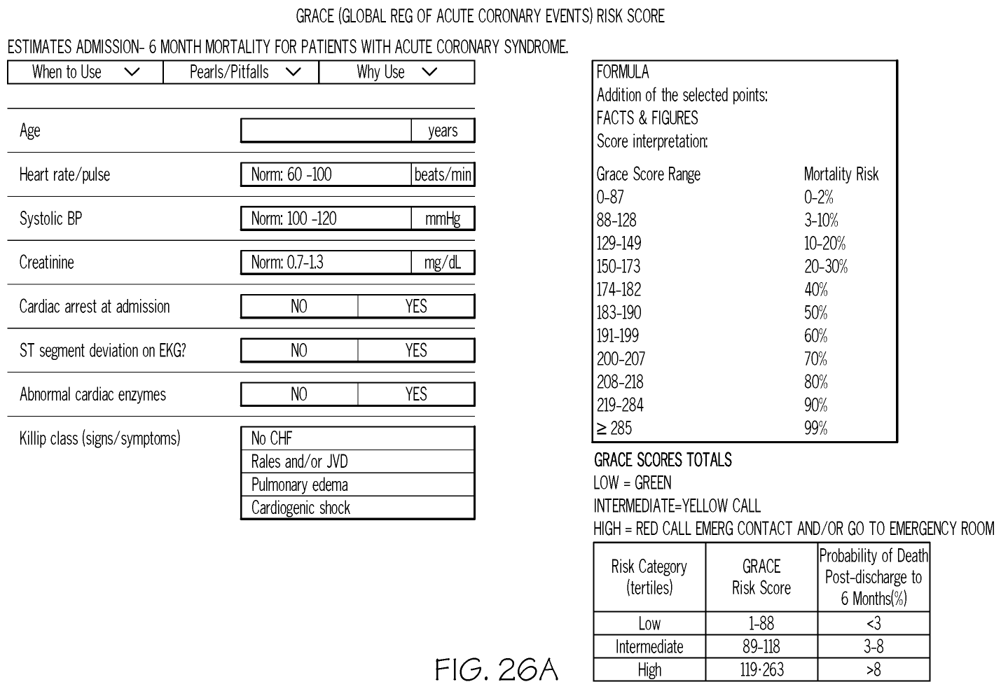

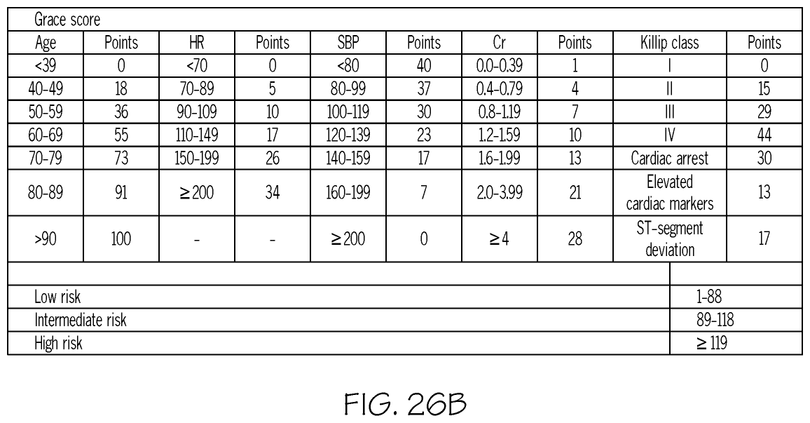

Examples are illustrated at FIGS. 26A and B. In the example of FIG. 26B, a low score: 1-88 can be processed by the smart watch 100 to display a green indicator (see HEART score for similar color matrix), an intermediate score 89-118 can be processed to display a yellow indicator. However, in addition, an automatic reporting mechanism is generated, e.g., phone call or text message to an emergency contact, etc. that reports this score. Similarly, a high score greater than 119 can be processed to display a red indicator along with a phone call or text message to the same or different contact than that of the intermediate score, for example, a 911 operator that reports this high score.

At block 508, a third special purpose computer processor 553 of the cardiac event risk assessment processor 209, referred to as a TIMI score processor, generates a TIMI score from a first set of input data. A TIMI risk score is a tool used in the medical community, in particular, emergency rooms, to predict the chances of having or dying from a heart event for people with angina, non-ST-segment elevation myocardial infarction (NSTEMI), or related heart conditions or types of heart attacks.

A TIMI score can range from 0-7, wherein one point is applied for the following: being older than 65, using aspirin within the last week, having at least two angina episodes in the last 24 hours, having elevated serum cardiac biomarkers, having ST-segment deviation on an ECG, having known coronary artery disease, having at least three risk factors for heart disease, which include: high blood pressure (greater than 140/90), smoking (being a current smoker), low HDL cholesterol (less than 40 mg/dL), diabetes, and/or a family history of heart disease. At least one of the foregoing is received as an input by a sensor of the smartwatch that is processed by a sensor processor 206, such as a blood pressure and/or ECG sensor processor.

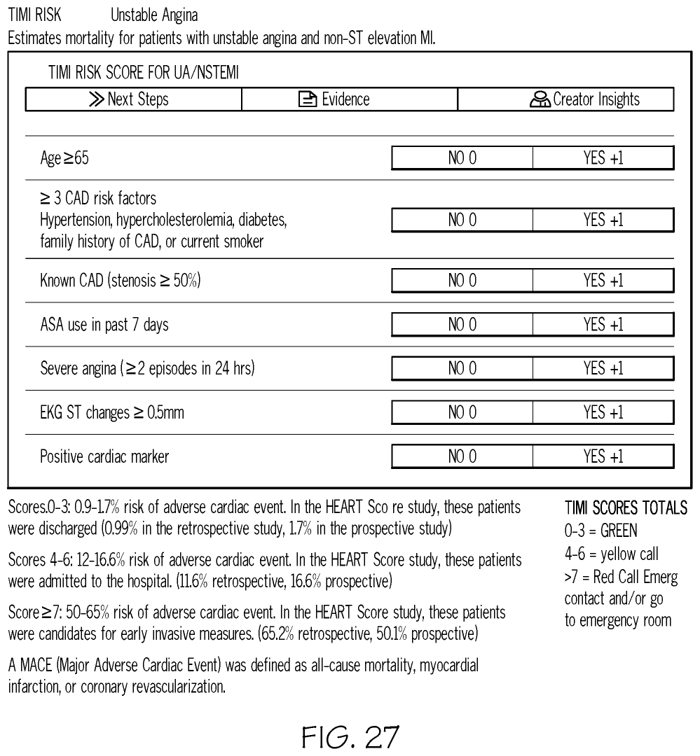

An example TIMI risk scoring matrix is illustrated at FIG. 27. A low score of a TIMI risk factor such as age, etc. of less than 2 can be processed by the smart watch 100 to display a green indicator (see HEART and GRACE scores for similar color matrix), an intermediate score less than 3 can be processed to display a yellow indicator. However, in addition, an automatic reporting mechanism is generated, e.g., phone call or text message to an emergency contact, etc. that reports this score. Similarly, a high score greater than 4 or more can be processed to display a red indicator along with a phone call or text message to the same or different contact than that of the intermediate score, for example, a 911 operator that reports this high score.

At decision diamond 510, a determination is made whether one or more of the scores determined at steps 5040, 506, and 508, respectively, exceeds a threshold value that can indicate a risk of an undesirable cardiac event. As described above, a HEART score can range from 0-2, a GRACE score can range from 0-363, and a TIMI score can range from 0-7. The threshold value can be at a higher end of each or a combination of these ranges. If the threshold value is exceeded, then the method 500 proceeds to block 512, where an action is performed to reduce the risk of the predicted cardiac event. Example actions can include but not be limited to the smart watch 100 automatically calling a hospital, 911 operator, or emergency contact, displaying warnings, and/or automatically opening the medication compartment 104 to present a life-saving pill or the like to the user. Otherwise, if the threshold value is not exceeded by the calculated scores, then the method 500 proceeds to block 514, where the scores and/or other assessment information are displayed from the smart watch 100.

Referring again to the cardiac event risk assessment processor 209 of FIG. 5A, each of the HEART score processor 551, GRACE score processor 552, and TIMI score processor 553 receives various inputs for generating the respective risk scores. Some of the inputs may be received from a same or common input. For example, all three processors 551, 552, 553 may receive data regarding user age ECG readings, and user inputs regarding lab data such as positive cardiac enzymes and proteins linked with heart muscle injuries, such as creatine phosphokillase (CPK), creatine kinase (CK), and troponin. For example, the HEART score processor 551 and TIMI score processor 553 may each receive the same input from an ECG sensor processor 206. User inputs, for example, historical data, may be input from a personal computer or a database or other computer of the network 16 shown in FIG. 3, e.g., internet computer 26, in addition to or instead of a user entering the data manually to the display 106 of the smart watch 100.

In some embodiments, a “super-score” can be generated from each of the HEART, GRACE, and TIMI scores. The system may apply a weight factor to each score, for example, 0.3, 0.3, and 0.4 respectively to generate the combined super score, which can be compared to a predetermined threshold comprising thresholds of the HEART, GRACE, and TIMI scores.

As described herein, in some embodiments, the smart watch 100 can output data to a medical personnel computer. Here, the data can be generated by the smart watch 100 in a suitable format for various medical software applications such as EMR modules, e.g., an outpatient pharmacy module provided via Epic Systems Willow, Eclinical Works, or an ambulatory module by Meditech and Cerner.

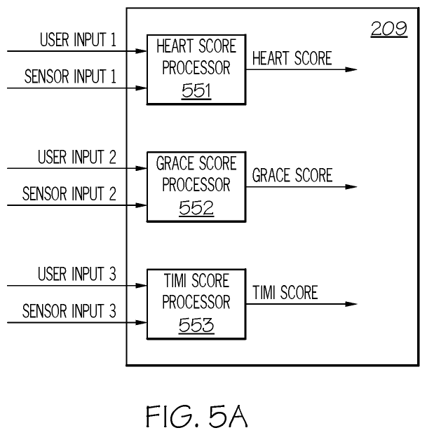

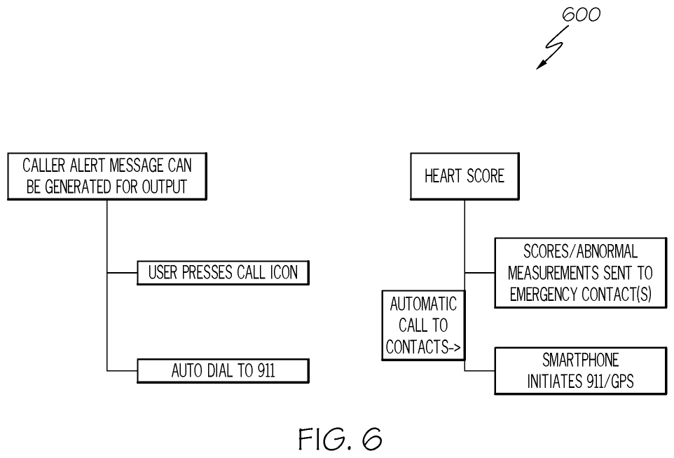

FIG. 6 illustrates a first use case where call or emergency contact is initiated from the smart watch 100 by selecting the call icon 402, or by an automatic call initiation. More specifically, in the flow diagram 600 of FIG. 6, a caller alert message can be generated and output to a local authority such as a police department, fire department, hospital, 911 service, and so on. A call can automatically be generated if one or more health scores, for example, shown in FIG. 5 exceeds a predetermined value, or if other abnormal health data is received and processed by the smart watch 100. In other embodiments, the call can be manually made by the user selecting the call icon 402 in response to viewing abnormal data or health scores, for example, which may be displayed in a particular color such as red, yellow, or green. In some embodiments, information in addition to a score or abnormal health information may be provided with the call such as a GPS location of the smart watch 100. The GPS processor 203 of the smart watch 100 can identify the location of the smart watch 100 which in turn can be used when sending and receiving communications to/from the smartphone 100 and an intended recipient, for example, an emergency contact, 911 operator, and so on.

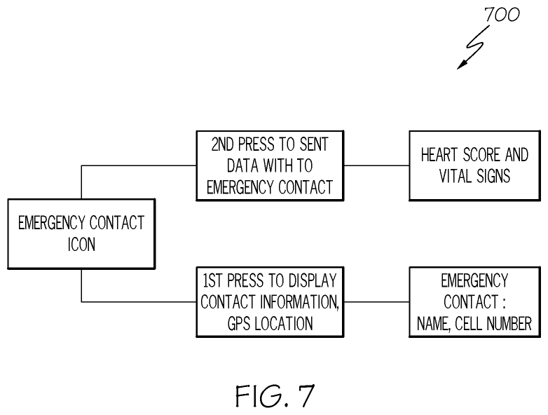

FIG. 7 illustrates a second use case where emergency information is retrieved and displayed by the smart watch 100. More specifically, in the flow diagram 700 of FIG. 7, emergency contact information and/or patent information such as abnormal health information, vital signs, and so on can be output to a predetermined cellphone or call-receiving apparatus 28 identified as an emergency contact. Here, the call icon 402 can operate as a 911 call button. When the call icon 402 is activated a first time, emergency contact information can be displayed. When activated a second time, the call is made by the smart watch 100 to the emergency contact stored in memory or contact database in the smart watch 100. In addition to the call, some or all of the foregoing information, e.g., health information, vital signs, etc., can be output to the emergency contact recipient.

FIG. 8 illustrates a third use case where vital sign information can be displayed by the smart watch 100. More specifically, in the flow diagram 800 of FIG. 8, vital sign information can be displayed in response to selecting a vital signs icon 403 from the display 106. Vital sign information may include blood pressure, heart rate, respiratory rate, abnormal measurements, and so on. In some embodiments, selecting the vital sign button 403 can in response output this data to a contact, for example, an emergency contact having a phone number, email address, or other contact information stored in a memory at the smart watch 100.

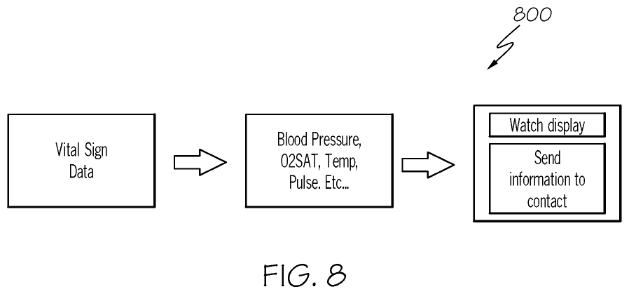

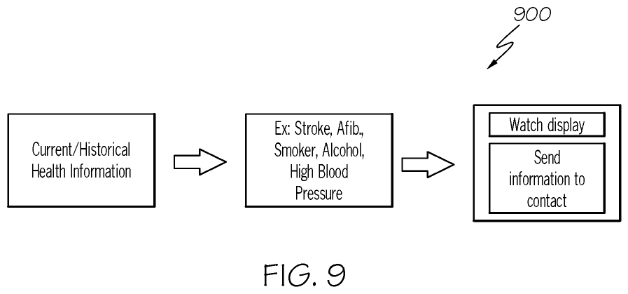

FIG. 9 illustrates a fourth use case where smartphone wearer health information can be displayed by the smart watch 100. More specifically, in the flow diagram 900 of FIG. 9, smartphone wearer health information can be displayed in response to selecting an icon from the display 106. Smartphone wearer health information may include current or historical data such as stroke, fibrillation, smoking or alcohol history, blood pressure, and so on. In some embodiments, selecting the button 403 can in response output this data to a contact, for example, an emergency contact having a phone number, email address, or other contact information stored in a memory at the smart watch 100.

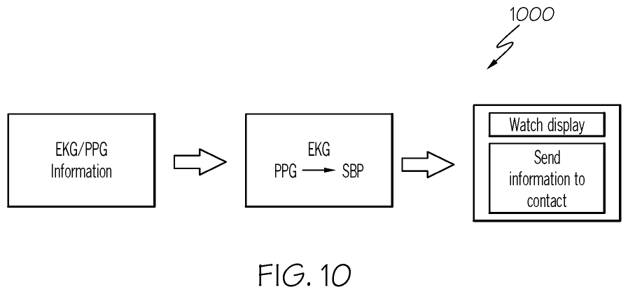

FIG. 10 illustrates a fifth use case where smartphone wearer ECG/PPG information can be displayed by the smart watch 100. More specifically, in the flow diagram 1000 of FIG. 10, ECG/PPG information can be displayed in response to selecting ECG/PPG icon 405 from the display 106. This may include graphical, textual, and or image data. In some embodiments, selecting the ECG/PPG icon 405 can in response output this data to a contact, for example, an emergency contact having a phone number, email address, or other contact information stored in a memory at the smart watch 100.

FIG. 11 illustrates a sixth use case where blood clot information concerning the wearer can be displayed by the smart watch 100. More specifically, in the flow diagram 1100 of FIG. 11, blood clot location information can be displayed in response to selecting a blood clot location icon 406 from the display 106. This may include graphical, textual, and or image data, for example, a display 1110 of the location of a blood clot shown in FIG. 11. In some embodiments, selecting the icon 406 can in response output this data to a contact, for example, an emergency contact having a phone number, email address, or other contact information stored in a memory at the smart watch 100.

With further regard to PPG signal data, PPG relies on IR light or the like which when traveling through biological tissue is absorbed by bones, skin pigments and both venous and arterial blood. Since light is more strongly absorbed by blood than the surrounding tissues, the changes in blood flow can be detected by PPG sensors as changes in the intensity of light. The voltage signal from a PPG result is proportional to the quantity of blood flowing through the blood vessels, which can provide for the detection of small changes in blood volume. A PPG signal has several components including volumetric changes in arterial blood which is associated with cardiac activity, variations in venous blood volume which modulates the PPG signal, a DC component showing the tissues' optical property and subtle energy changes in the body. Major factors affecting the recordings from the PPG can include a location of measurement and the contact force between the location and the sensor.

The smart watch 100, unlike conventional smart watches including PPG as well as ECG detection features, applies the foregoing to detect a low blood flow as an indicator of a new possible stroke for users with a previous stroke in combination with user data processed by the cardiac event risk assessment processor 209. The smart watch 100 can therefore include sensors that include high-intensity green light-emitting diodes (LEDs) and photodetectors that help reliable monitoring of the pulse rate in a non-invasive manner, and sensors that monitor minor variations in the intensity of light transmitted through or reflected from the tissue. These intensity changes are associated with changes in blood flow through the tissue and provide vital cardiovascular information such as the pulse rate.

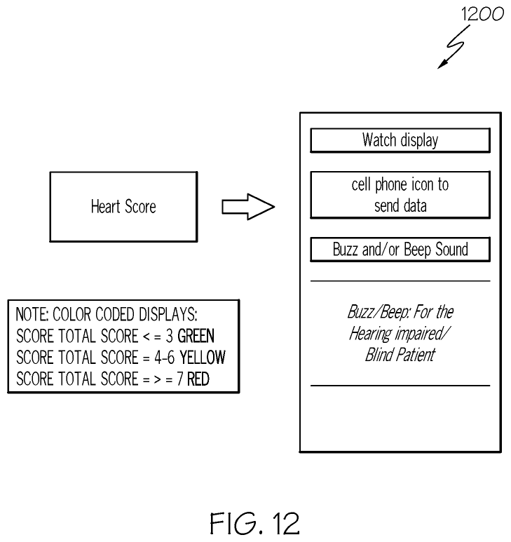

FIG. 12 illustrates a seventh use case where a health score concerning the wearer can be displayed by the smart watch 100. More specifically, in the flow diagram 1200, HEART scores are managed so that a score of 0-3 indicates a 0.9-1.7% risk of cardiac event, whereby the smart watch 100 translates the score to a particular display, for example, a green score or graphic or other indicator in audio, tactile, and/or visual format. A HEART score of 4-6 indicates a 12-16.6% risk of an adverse cardiac event, where a different indicator, for example, a yellow indicator, is displayed. Similarly, a high risk score of 7 or more indicates a 50-65% of an adverse cardiac event, possibly, a major event causing mortality or major cardiovascular damage, where a clear audio, tactile, and visual communication, e.g., red flashing warnings, automatic phone call to a 911 operator, and/or other action is performed.

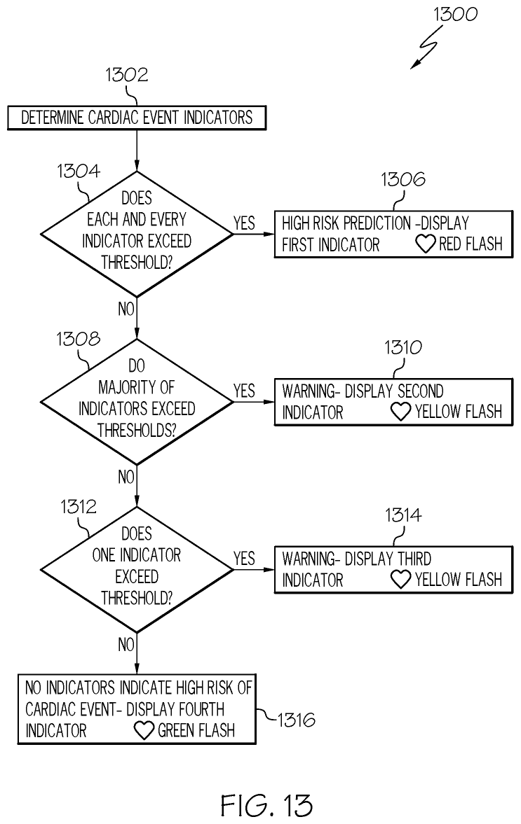

FIG. 13 is a flow diagram of a method 1300 for assessing a cardiac event, in accordance with some embodiments. In describing the method 1300, reference is made to elements of FIGS. 1-12. Accordingly, some or all of the method 500 can be performed by elements of FIGS. 1-12.

At block 1302, three different indicators are provided as evidence of a possible cardiac event, and that form a tri-measurement determination. In some embodiments, the three indicators are a HEART score, GRACE score, and TIMI risk score, but not limited thereto. The indicators are determined by a combination of sensor and user inputs to the smart watch 100, as described above.

At decision diamond 1304, a determination is made whether each and every indicator (e.g., all three indicators: HEART, GRACE, TIMI scores) exceeds a predetermined threshold. For example, the threshold value for a HEART score is “0”. If the HEART score is determined to be +1 (indicating moderate risk) or a +2 (indicating high risk), then the score is stored in memory. Similarly, the threshold value for a GRACE score is “200” (range is 0-363). If the GRACE score is greater than 200, then the GRACE score is stored in memory with the HEART score. Similarly, the threshold value of a TIMI score is 4. If the TIMI score is greater than 4, then the score is stored in memory with the HEART and GRACE scores. In the foregoing example, since all three scores exceed their respective threshold values, the method 1300 proceeds to block 1306, where a first display indicator is generated that indicates a high risk potential of an adverse cardiac event.

In some embodiments, at block 1306, the first display indicator displayed on the smart watch 100, may include a heart shaped button or other electronic display symbol, shape, or the like that is displayed as a red heart of the like, and flashes or otherwise displayed to warn the viewer of the possibility of a cardiac event. In some embodiments, a message will inform the user and automatically notify (via peripheral I/O devices of the smart watch) emergency contacts (beep/vibrate/text, call 911 and emergency contact) of possible a heart event. The display 106 may display function keys for permitting a user to select them to view abnormal measurements from the HEART, GRACE, and/or TIMI Risk scores or related details. In some embodiments, actions can be reset after and incoming call/text response acknowledgement has occurred.

If at decision diamond 1304, a determination is made that not all three indicators exceed a threshold value, also referred to as a risk threshold, then the method 1300 proceeds to decision diamond 1308, where a determination is made whether a majority, for example, two of three indicators exceeds a predetermined threshold, or whether there is a significant change in one or more vital signs, such as blood pressure, ECG, or PPT as determined by a smart watch sensor. If yes, then the method 1300 proceeds to block 1310, where a second display indicator is generated that indicates a warning of an adverse cardiac event.

In some embodiments, at block 1310, the second display indicator displayed on the smart watch 100 may include a heart shaped button or other electronic display symbol, shape, or the like that is displayed as a yellow heart or the like (different than the displayed indicator in step 1306), and flashes or otherwise displayed to warn the viewer of the possibility of a cardiac event. Also, a score can be displayed, for example, a “2” value inside or otherwise part of the yellow heart. In some embodiments, a message will inform the user and automatically notify (via peripheral I/O devices of the smart watch) emergency contacts (beep/vibrate/text, call 911 and emergency contact) of a possible event. Other information such as abnormal measurements can be output as well. The yellow flashing symbol or the like can be discontinued when the reset button is pressed. The display 106 may display function keys for permitting a user to select them to view abnormal measurements from the HEART, GRACE, and/or TIMI Risk scores or related details shown in the examples of FIGS. 25-27, contents of which may be stored at the cloud database 24, a portion of memory of the smart watch 207, or a combination thereof. In some embodiments, actions can be reset after and incoming call/text response acknowledgement has occurred.

If at decision diamond 1308, a determination is made that that the majority of indicators does not exceed a threshold value, then the method 1300 proceeds to decision diamond 1312 where a determination is made whether a single indicator exceeds a predetermined threshold, or whether there is a significant change in one or more vital signs, such as blood pressure, ECG, or PPT as determined by a smart watch sensor. If yes, then the method 1300 proceeds to block 1314, where a third display indicator is generated that indicates a warning of an adverse cardiac event.

In some embodiments, at block 1314, the first display indicator displayed on the smart watch 100, may include a heart shaped button or other electronic display symbol, shape, or the like that is displayed as a yellow heart or the like (different than the displayed indicator in step 1306), and flashes or otherwise displayed to warn the viewer of the possibility of a cardiac event. Also, a score can be displayed, for example, a “1” value (different than the value generated at block 1310) inside or otherwise part of the yellow heart. In some embodiments, a message will inform the user and automatically notify (via peripheral I/O devices of the smart watch) emergency contacts (beep/vibrate/text, call 911 and emergency contact) of a possible event. Other information such as abnormal measurements can be output as well. The yellow flashing symbol or the like can be discontinued when the reset button is pressed The display 106 may display function keys for permitting a user to select them to view abnormal measurements from the HEART, GRACE, and/or TIMI Risk scores or related details. In some embodiments, actions can be reset after and incoming call/text response acknowledgement has occurred.

With further regard to a yellow display, the medication compartment can store medication and if the compartment is opened, the yellow alert and corresponding risk score can be sent via text/email to the user's emergency contact.

If at decision diamond 1312, a determination is made that that at least one indicator does not exceed a threshold value, then the method 1300 proceeds to block 1316 where a fourth indicator such as a green heart is displayed indicating no high risk of an adverse cardiac event.

As described in FIG. 13, various displays can be presented in yellow, red, or green depending on a score result indicating varying degrees of risk of an adverse cardiac event. The color may change in real-time if different inputs are received. For example, one score may be generated in response to a sensor indicating a safe ECG level. However, the sensor may later receive signals indicating a life-threatening ECG level, which may automatically change one or more indicator scores, and also result in an automatic change in display indicator color, for example, from green to red. This may include a feature of the sensor processor 206 continuously measure a user's physiological status.

In summary, in method 1300, one risk score of the HEART, GRACE, and TIMI scores exceeding its threshold value can trigger the smart watch to automatically notify the wearer via its display and/or other contacts identified in a contact directory in the smart watch.

Two risk scores exceeding their respective threshold values can elevate the risk of a cardiac event and therefore, the smart watch can display a heightened alert in combination with other outputs, such as beeps (for blind users), vibrations (for hearing-impaired users), and so on, and send the score data to the predetermined contact(s). If all three scores exceed their respective threshold values, then the smartphone can automatically call a 911 number, or present heightened alarms for the user.

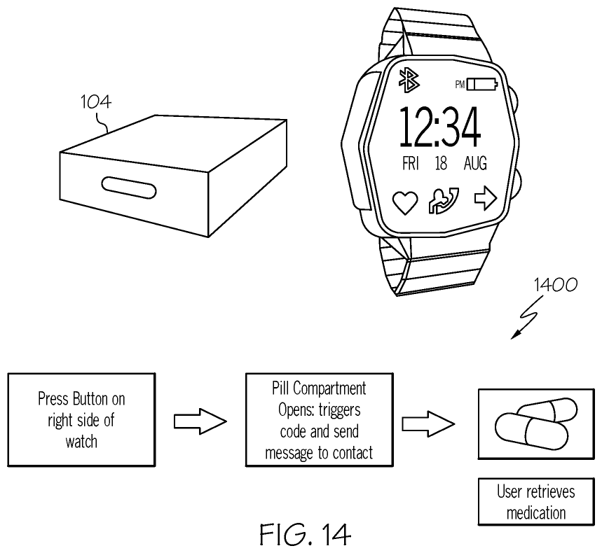

FIG. 14 is a flow diagram 1400 of an operation performed by a smart watch in response to the selection of a medication dispensing button displayed at the smart watch, in accordance with some embodiments. More specifically, pressing a release button 105 opens the medication compartment 104. In addition, selecting the button 105 can in response result in a call made to emergency contact having a phone number, email address, or other contact information stored in a memory at the smart watch 100, wherein a message can be included, for example, stating that the medication has been dispensed.

As will be appreciated by one skilled in the art, aspects of the present invention may be embodied as a system, method, or computer program product. Accordingly, aspects of the present invention may take the form of an entirely hardware embodiment, an entirely software embodiment (including firmware, resident software, micro-code, etc.), or an embodiment combining software and hardware aspects that may all generally be referred to herein as a “service,” “circuit,” “circuitry,” “module,” and/or “system.” Furthermore, aspects of the present invention may take the form of a computer program product embodied in one or more computer readable medium(s) having computer readable program code embodied thereon.

Any combination of one or more computer readable medium(s) may be utilized for various functions of the smart watch, such as processing, signaling, display data, measurement, sound, image, or video generation, display light control, and so on.

The computer readable medium may be a non-transient computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Program code and/or executable instructions embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer (device), partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described herein with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, and/or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

While the invention has been described with reference to certain embodiments, it will be understood by those skilled in the art that various changes may be made and equivalents may be substituted for elements thereof to adapt to particular situations without departing from the scope of the disclosure. Therefore, it is intended that the claims not be limited to the particular embodiments disclosed, but that the claims will include all embodiments falling within the scope and spirit of the appended claims.

What is claimed is:

1. A clinical smart watch, comprising:
a housing;
a band coupled to the housing, at least one of the housing or the band including one or more sensors that measure health-related events of a wearer of the smart watch;
a cardiac event risk assessment processor in the housing that receives and processes sensor signals corresponding to the measured health-related events;
a user interface that includes an input for providing user input data to the cardiac event risk assessment processor, wherein the cardiac event risk assessment processor executes a prediction model including a first risk score generated from a first combination of the user input data and the sensor signals, a second risk score generated from a second combination of the user input data and the sensor signals, and a third risk score generated from a third combination of the user input data and the sensor signals;
a peripheral device for initiating an electronic communication with a remote electronic device when at least one of the first, second, or third risk scores exceeds a threshold value indicating a probability of an adverse cardiac event;
a distress button that activates the peripheral device to initiate the electronic communication with the remote electronic device;
a medication compartment that stores a source of medication for treating the adverse cardiac event and that opens by separating from the housing to expose the source of medication; and
a display for displaying each of the first, second, and third risk scores, and for generating a visual alert when the at least one of the first, second, or third risk scores exceeds the threshold value.

2. The clinical smart watch of claim 1, wherein the first risk score is a HEART (History, ECG (EKG), Age, Risk actors, and Troponin) score, the second risk score is a GRACE (Global Registry of Acute Coronary Events) score, and the third risk score is a TIMI (Thrombolysis In Myocardial Infarction) score.

3. The clinical smart watch of claim 1, further comprising a memory that stores and exchanges a combination of the user input data and sensor signal data and log reports including historical alarm data, patient data, and electronic messages, and health status information with a remote cloud data repository.

4. The clinical smart watch of claim 1, wherein the cardiac event risk assessment processor generates from the first, second, and third risk scores a risk assessment of a possible cardiac event, wherein the risk assessment in response is used to determine an action directed at prevent an occurrence of a dangerous cardiac event.

5. The clinical smart watch of claim 1, wherein the user interface displays on the display a heart alert icon, a call icon, a vital signs icon, a problems icon, an ECG/PPG icon, and a clot icon.

6. The clinical smart watch of claim 1, wherein the cardiac event risk assessment processor further automatically establishes an electronic communication with a remote electronic device to provide alert notification information when the at least one of the first, second, or third risk scores exceeds the threshold value.

7. The clinical smart watch of claim 1, wherein the first, second third combinations of the user input data and the sensor signals are generated by at least one common sensor and at least one common user input.

8. The clinical smart watch of claim 1, wherein the medication compartment transitions from a closed state to an open state relative to the housing to expose the source of medication when the at least one of the first, second, or third risk scores exceeds the threshold value

9. A clinical smart watch, comprising:
one or more computing devices; and
a non-transitory memory medium that stores instructions that when executed by the one or more computing devices executes the clinical smart watch to perform at least one operation comprising the steps of:
collecting tri-measurement score data from a combination of sensors, health status repository, and user inputs;
generating from the tri-measurement score data a tri-measurement score, comprising:
generating a HEART (History, ECG (EKG), Age, Risk actors, and Troponin) risk score;
generating a GRACE (Global Registry of Acute Coronary Events) score; and
generating a TIMI (Thrombolysis In Myocardial Infarction) score;
comparing the tri-measurement score to a threshold value; and
generating a cardiac event assessment notification in response to a result of the comparison of the tri-measurement score and the threshold value.

10. The clinical smart watch of claim 9, wherein the tri-measurement score data includes a combination of real-time sensor data, historical data, medical opinions, age, risk factors, and/or lab results.

11. The clinical smart watch of claim 9, wherein the memory stores and exchanges a combination of the user input data and sensor signal data and log reports including historical alarm data, patient data, and electronic messages, and health status information with a remote cloud data repository.

12. The clinical smart watch of claim 9, wherein the one or more computer processors processes from the HEART, GRACE, and TIMI scores a risk assessment of a possible cardiac event, wherein the risk assessment in response is used to determine an action directed at prevent an occurrence of a dangerous cardiac event.

13. The clinical smart watch of claim 9, wherein the one or more computing devices executes the clinical smart watch to further generate for a display a heart alert icon, a call icon, a vital signs icon, a problems icon, an ECG/PPG icon, and a clot icon.

14. The clinical smart watch of claim 9, wherein the wherein the one or more computing devices executes the clinical smart watch to further automatically establish an electronic communication with a remote electronic device to provide alert notification information when the at least one of the tri-measurement score exceeds the threshold value.

15. The clinical smart watch of claim 9, wherein the user input data and the sensor signals are generated by at least one common sensor and at least one common user input.

16. A clinical smart watch, comprising:
one or more computing devices; and
a non-transitory memory medium that stores instructions that when executed by the one or more computing devices executes the clinical smart watch to perform at least one operation comprising the steps of:
determining a plurality of cardiac indicators, including:
generating a first user interface display in response to a determination that all of the cardiac indicators exceed a risk threshold;
generating a second user interface display in response to a determination that a majority of the cardiac indicators exceed a risk threshold;
generating a third user interface display in response to a determination that a minority of the cardiac indicators exceed a risk threshold; and
generating a fourth user interface display in response to a determination that none of the cardiac indicators exceeds a risk threshold.

17. The clinical smart watch of claim 16, wherein the memory stores and exchanges a combination of the user input data and sensor signal data and log reports including historical alarm data, patient data, and electronic messages, and health status information with a remote cloud data repository.

18. The clinical smart watch of claim 16, wherein the one or more computer processors processes from the cardiac indicators a risk assessment of a possible cardiac event, wherein the risk assessment in response is used to determine an action directed at prevent an occurrence of a dangerous cardiac event.

19. The clinical smart watch of claim 16, wherein the one or more computing devices executes the clinical smart watch to further generate for a display a heart alert icon, a call icon, a vital signs icon, a problems icon, an ECG/PPG icon, and a clot icon.

20. The clinical smart watch of claim 16, wherein the wherein the one or more computing devices executes the clinical smart watch to further automatically establish an electronic communication with a remote electronic device to provide alert notification information when only the majority or all of the cardiac indicators exceeds the threshold value.
